(19) United States
(12) Patent Application Publication
(10) Pub. No.: US 2023/0241222 A1
(43) Pub. Date: Aug. 3, 2023

(54) PHARMACEUTICAL COMPOSITION FOR SARS-COV-2 PREVENTION AND/OR TREATMENT

(71) Applicant: Ahmed Abdelaal AHMED MAHMOUD METWALLY ALKHATIP, Birmingham (GB)

(72) Inventor: Ahmed Abdelaal AHMED MAHMOUD METWALLY ALKHATIP, Birmingham (GB)

(21) Appl. No.: 18/009,725

(22) PCT Filed: Jun. 22, 2020
(86) PCT No.: PCT/IB2020/055863

§ 371 Date: Dec. 11, 2022

(51) Int. Cl. A61K 47/52

(52) U.S. Cl. CPC A61K 47/52

(57) ABSTRACT

The present invention provides a pharmaceutical composition comprising a compound for use in preventing and/or treating infections caused by SARS-CoV2. There is also provided, the use of a pharmaceutical composition comprising a compound for the manufacture of a medicament for the prevention and/or the treatment of infections caused by SARS-CoV-2. Besides that, the present disclosure relates also to a method for inhibiting the production of SARS-CoV2. Provided, a method for virtual screening for such compounds.

### FIELD OF THE INVENTION

This disclosure relates to medicine. In particular this disclosure relates to the prevention and/or the treatment of infections caused by SARS-CoV-2.

### BACKGROUND

Since November 2019, the novel coronavirus known as SARS-CoV-2 had an universal impact in terms of economic dislocation, the burden on local and global public health. The current pandemic has ravaged communities and overwhelmed medical facilities worldwide and induced more than 7.273.958 cases and 413.372 deaths globally as of Jun. 12, 2020.

The effect of the new coronavirus on society and the global economy is unprecedented and gaved rise to urgent research for effective therapeutic strategies against SARS-CoV-2.

Although several anti-virals and agents have shown antiviral activity against SARS-CoV-2, at present there are no antiviral therapies of validated efficiency in treating patients with SARS-COV-2. For instance, the FDA (food and drugs administration) in USA issued on 27 Mar. 2020 an EUA (Emergency use authorization) for emergency use of Remdesivir for the treatment of hospitalized 2019 coronavirus disease (SARS-COV-2) patients. Remdesivir is a direct acting antiviral drug that inhibits viral RNA synthesis. It is an investigational drug and is not currently approved for any indication.

Thus, there is a need for effective compositions and methods for preventing and/or treating SARS-CoV-2. The present invention addresses these and other needs.

### SUMMARY OF THE INVENTION

The present invention provides a pharmaceutical composition comprising a therapeutically effective amount of a metal complexed Methisazone or a pharmaceutically acceptable salt thereof, and optionally a pharmaceutically acceptable carrier or excipient; for use in the prevention and/or treatment of SARS-CoV-2 infections.

There is also provided, the use of a pharmaceutical composition comprising a therapeutically effective amount of a metal complexed Methisazone or a pharmaceutically acceptable salt thereof, and optionally a pharmaceutically acceptable carrier or excipient for the manufacture of a medicament for the treatment and/prevention of SARS-CoV-2 infections.

Besides, the present disclosure provides a method for inhibiting the production of SARS-CoV2 comprising treating a human infected with SARS-CoV2 with a pharmaceutical composition comprising a therapeutically effective amount of metal complexed Methisazone or a pharmaceutically acceptable salt thereof, and optionally a pharmaceutically acceptable carrier or excipient.

The composition or method may optionally comprise one or more additional anti-viral agents.

Some embodiments of the invention relate to the method above, further including a step of virtually screening a library of molecules for a molecule that is predicted to bind to or interact with at least one of the amino acids of SARS-CoV-2.

The present invention is based, in part, on certain discoveries which are described more fully in the ‘Materials and Methods’ section of the present application. For example, the present invention is based, in part, on the discovery that the Methisazone displays better binding energies to Sars-Cov-2 proteins in its Fe-complexed form.

The details of one or more embodiments of the invention are set forth in the description below. Other features, objects, and advantages of the invention will be apparent from the description and the claims.

Still other objects and advantages of the invention will become apparent to those of skill in the art from the disclosure herein, which is simply illustrative and not restrictive. Thus, other embodiments will be recognized by the skilled artisan without departing from the spirit and scope of the invention.

### BRIEF DESCRIPTION OF THE FIGURES

FIG. 1: shows SARS-CoV-2 replication process

FIG. 2: shows the molecular structure of Methisazone and its two isomeric states.

FIG. 3: shows a Methisazone, Fe-Methisazone and Zn-Methisazone docked with SARS-CoV-2 proteins main protease, RNA dependent RNA polymerase, spike protein, papain-like protease (PLPro).

FIG. 4: shows the Fe-Methisazone docked with the RNA-dependent RNA polymerase (RDRP) of Sars-CoV-2.

FIG. 5: shows the energetic contribution of the metal atom vs other contributions to the binding energy in Fe-Methisazone and Zn-Methisazone dockings.

FIG. 6: shows the free Methisazone predicted binding energies distribution, free Methisazone predicted binding energies distribution. The binding energy ranges are in the X axis, whereas the range frequencies are in the Y axis.

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention.

### DETAILED DESCRIPTION

### Definitions

Unless stated otherwise, the following terms and phrases as used herein are intended to have the following meanings:

When trade names are used herein, applicants intend to independently include the trade name product and the active pharmaceutical ingredient(s) of the trade name product. As used herein, “a compound of the invention” means a compound of Formula metal-Methisazone or a pharmaceutically acceptable salt, thereof.

“Effective amount” as generally used herein refers to an amount, or dose, within the range normally given or prescribed to demonstrate an anti-viral effect, e.g., in vitro or in vivo. The range of an effective amount may vary from individual to individual; however, the optimal dose is readily determinable by those of skill in the art depending upon the use. Such ranges are well established in routine clinical practice and will thus be readily determinable to those of skill in the art.

“Pharmaceutically acceptable” as generally used herein refers to those compounds, materials, compositions, and/or dosage forms which are, within the scope of sound medical judgment, suitable for use in contact with the tissues of human beings without excessive toxicity, irritation, allergic response, or other problems or complications commensurate with a reasonable benefit/risk ratio.

The terms “treating or preventing” are intended to include preventing, eradicating, or inhibiting the viral infection, for example, in the context of the therapeutic or prophylactic methods of the invention.

It is an object of the present invention to provide an effective solution to treat coronavirus sars 2 infection.

Reference will now be made in detail to certain embodiments of the invention, examples which are illustrated in the accompanying description, structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention.

### SARS-CoV-2 Replication

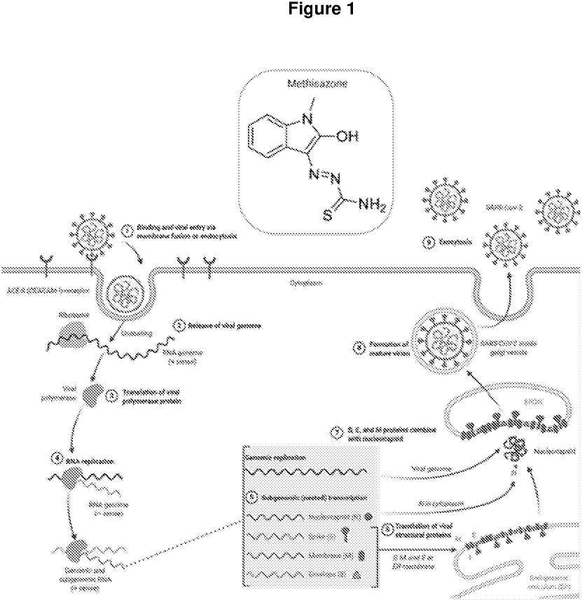

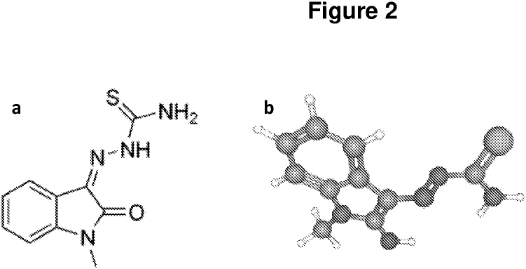

A key to curbing SARS-CoV-2 Is to understand how it enters cells.With reference to FIG. 1 In fact, SARS-CoV-2 can enter the human body through its receptors, angiotensin-converting enzyme-II (ACE-II), which are found in several sites, including lungs, heart, kidneys, and gastrointestinal tract, thus facilitating viral entry into host cells. The viral entry starts by the attachment of the spike protein to ACE-II receptor in the target cells (for instance, nasal epithelial cells which have the highest ACE-II expression throughout the respiratory tract)1,2. This attachment occurs in the binding domain of spike glycoprotein of the receptor3. The entry and binding processes are then followed by fusion of the viral membrane and host cell4. When virions are taken up into endosomes, cathepsin L activates the spike protein. Alternatively, the spike protein can be activated by the cellular type II transmembrane serine protease (TMPRSS2) which is near to the ACE-II receptor on the surface of the target cell, TMPRSS2 could clear the ACE-II as well after fusion occurs 1. Of note, activation of the spike glycoproteins leads to conformational changes that lead to the fusion of viral envelope protein with target cell membrane following entry via endosomal pathway5,6.

### Translation, Replication and Transcription

Following viral entry, the release of viral RNA into the host cytoplasm occurs that undergoes translation and generates replicase polyproteins pp1a and pp1b that further cleaved by SARS-CoV-2 encoded proteases into smaller proteins. The replication of SARS-CoV-2 involves ribosomal frameshifting during the translation process and generates both genomic and several copies of subgenomic RNA by discontinuous transcription that encodes for relevant viral proteins7.

### Assembly and Release

Virions assembly takes place via interaction of viral RNA and S1, S2, envelope, and membrane structural proteins, translated by ribosomes, at the endoplasmic reticulum and Golgi complex. The nucleocapsid remains in cytoplasm and is assembled from genomic RNA. They fuse with the virions precursor, which is then transported from the ER through the Golgi apparatus to the cell surface via small vesicles. Virions are subsequently released from the infected host cells via vesicles and search for new target cell8.

Various researches have been carried out to find an antiviral for the treatment of the new Sars-CoV-2. lt is an object of the present invention to provide a composition for use in preventing and/or treating infections caused by SARS-CoV2.

In a first aspect of the present invention there is provided a pharmaceutical composition comprising a therapeutically effective amount of a metal complexed Methisazone or a pharmaceutically acceptable salt thereof, and optionally a pharmaceutically acceptable carrier or excipient; for use in the prevention and/or treatment of SARS-CoV-2 infections. In an embodiment the metal complexed Methisazone is an iron complexed Methisazone. In another embodiment the metal complexed Methisazone is a zinc complexed Methisazone.

In another aspect of the present invention there is provided the use of a pharmaceutical composition comprising a therapeutically effective amount of a metal complexed Methisazone or a pharmaceutically acceptable salt thereof, and optionally a pharmaceutically acceptable carrier or excipient for the manufacture of a medicament for the treatment and/prevention of SARS-CoV-2 infections.

In an embodiment the metal complexed Methisazone is an iron complexed Methisazone.

In another embodiment the metal complexed Methisazone is a zinc complexed Methisazone.

In a third aspect, this invention features a method for inhibiting the production of SARS-CoV2 comprising treating a human infected with SARS-CoV2 with a pharmaceutical composition comprising a therapeutically effective amount of metal complexed Methisazone or a pharmaceutically acceptable salt thereof, and optionally a pharmaceutically acceptable carrier or excipient.

In an embodiment the metal complexed Methisazone is an iron complexed Methisazone.

In another embodiment the metal complexed Methisazone is a zinc complexed Methisazone.

The antiviral activity of a compound of the invention can be measured using standard screening protocols that are known.

The compounds described above include the compounds themselves, as well as their salts, their solvates, and their prodrugs, if applicable.

The term “treatment” refers to therapeutic treatment as well as prophylactic or preventative measures to cure or halt or at least retard disease progress.

In fact, the Methisazone (USAN) or metisazone(INN) is an antiviral drug that works by inhibiting mRNA and protein synthesis. It has been used in the past to treat smallpox. Its molecular structure is displayed in the Erreur! Source du renvoi introuvable., where its two isomeric states are displayed.

That isomery is a keto-enol isomerism inside one of its cycles, and a similar isomerism in the side chain, where the thioketonic atom switches double bond switches between the sulfur atom and the middle nitrogen atom. The electronic orbitals can be shared not only among that atoms, but there exist coordination complexes of that part of the Methisazone to several metals: iron, copper, zinc and cobalt.

In the context of this worldwide emergency from SARS-COV-2, we wanted to predict if Methisazone, free or in complex with metal, can show inhibitory activity to SARS-CoV-2. Thus, we executed a comprehensive molecular docking study to several proteins of the virus such as the main protease (which cuts the polyproteins translated from RNA to produce functional proteins), the RNA-dependent RNA polymerase (RDRP, responsible for RNA replication), the spike protein (responsible for viral entry) and the papain-like protease (responsible for processing three cleavage sites of the polyprotein to release mature non-structural proteins and has a deubiquitinase and delSGylating activity as well)12. Comparing with other inhibitors, remdesivir has calculated binding energy of -8.28 ±0.65 kcal/mol with RDRP13. That binding energy was calculated using molecular dynamics and free energy perturbation. Here, Methisazone displays better binding energies to SARS-CoV-2 proteins in its Fe-complexed form, with the better binding result at -17.46 kcal/mol versus the RDRP, at -17.41 kcal/mol of Fe-Methisazone versus the spike protein, and -15.96 of Fe-Methisazone versus papain-like protease. The better result that lay near the positions predicted by the PARS server to be an allosteric site or a binding pocket is for the Fe-Methisazone versus the RDRP, with predicted binding energy of -15.02 kcal/mol.

Preferably, the binding molecules, i.e. Fe-Methisazone, are capable of specifically binding to SARS-CoV-2. The composition can be administered to a human to treat, prevent or ameliorate one or more symptoms associated with a Sars-CoV-2 infection.

Also within the scope of this invention, a synergistic composition, which has a synergistic SARS-CoV-2 neutralizing activity as defined in the appended claims. In other words, the composition comprises at least the Methisazone complexed with iron and it is capable of specifically binding to a SARS-CoV-2 and that has neutralizing activity against SARS-CoV-2.

The compounds of the present invention can be used in the treatment of a human already suffering from a SARS-CoV-2, or can be administered prophylactically to reduce or prevent the chance of a SARS-CoV-2 infection.

In an embodiment the binding molecule Fe-Methisazone acts against the SARS-CoV-2 entry into the human cell, by specifically binding to surface accessible proteins of a SARS-CoV-2 which include, but are not limited to, inner and outer membrane proteins, proteins adhering to the cell wall, and potential secreted proteins. Relevant proteins of SARS-CoV2 in that respect, are among, the spike (S) protein, the membrane (matrix) protein, the (small) envelope. This mechanism can be the key mechanism for prophylaxis treatment against SARS-CoV-2.

That binding energy was calculated using molecular dynamics and free energy perturbation. Here we have that the methizazone displays better binding energies to Sars-Cov-2 proteins in its Fe-complexed form, with the better binding result at -17.46 kcal/mol vs the RDRP, but this molecule resulted at a different place than the remdemisvir. There were 3 docking results that resulted in a similar position than the remdemsivir. All the positions had the higher-affinity atom, the iron (of around -10 kcal/mol), the iron, making a salt bridge to the side chain of Asp760 of the RDRP. The other atoms resulted in very different positions. The better ranked molecule (-15.02 kcal/mol, 7th place) resulted in contact with the side-chain of Tyr619, asp618 and Trp617, the sidechain of Asp618 and the CB atom of Cys622. Other (20th place) has contact with CG2 of Thr687, the mainchain of Ala688, the OH and O of Ser759, the mainchain and sidechain of Asp761 (included a salt bridge). The other one (28th place) has contacts (with salt-bridge) to Asp623 and to Asp760, to Cys622 mainchain and sidechain, Tyr619 and Pro620 mainchain and Asp618 sidechain (this time without salt bridge). The zinc-bound Methisazone resulted with a result similar to the conformations of Fe-Methisazone best docking places 7 and 20, but with smaller binding energies, but none of the free Methisazone docking results were in similar position to the Fe-Mtz best docking places 7, 20 and 28; this can be taken as an evidence that the metal guides the Methisazone to a binding site, with the iron being the best binder.

In one embodiment Both Zn-Methisazone and Fe-Methisazone can be used together to treat SARS-CoV-2 to give better results.

In an embodiment, the Fe-Methisazone acts against the SARS-CoV-2 Virus replication inside the human cell by binding to the RNA-dependent RNA polymerase of SARS-CoV-2. The amino acid sequence of proteins of SARS-CoV-2, can be found in the GenBank EMBL-database and/or other databases.

In another embodiment, Fe-Methisazone can be combined with other antivirals to give better effects in treating SARS-CoV2 virus. (Justin Stebbing, Anne Phelan, Ivan Griffin, Catherine Tucker, Olly Oechsle, Dan Smith et al. SARS-COV-2: combining antiviral and anti-inflammatory treatments. VOLUME 20, ISSUE 4, P400-402, APRIL 01, 2020. DOI:https://doi.org/10.1016/S1473-3099(20)30132-8)

### Routes of Administration

Within the scope of the present invention, there is provided a method for inhibiting the production of SARS-CoV2 comprising treating a human infected with SARS-CoV2 with a pharmaceutical composition comprising a therapeutically effective amount of metal complexed Methisazone or a pharmaceutically acceptable salt thereof, and optionally a pharmaceutically acceptable carrier or excipient.

In one embodiment, the metal complexed Methisazone is an iron complexed Methisazone.

In another embodiment, the metal complexed Methisazone is a zinc complexed Methisazone.

Methisazone was reported to be administrated orally where adults receive 6 gm (four capsules of Marboran) a day, taken 3 gm in the morning and 3 gm in the evening while the dose is halved for children between 3 and 10 years old and proportionately reduced still further for infants14. Also, it was reported to be administrated at 200 or 600 mg/kg of body weight15. Mostly, Fe-Methisazone will be used with the same route of administration and possibly the same dose. However, these will be subject to clinical experiments following manufacturing of the Fe-Methisazone.

To practice the method of this invention, the above-described pharmaceutical composition can be administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. It will be appreciated that the preferred route may vary with for example the condition of the recipient.

In the method of the present invention for the treatment of SARS-CoV-2 the compounds of the present invention can be administered at any time to a human who may come into contact with humans infected by SARS-CoV-2. In some embodiments, the compounds of the present invention can be administered prophylactically to humans coming into contact with humans suffering from SARS-CoV-2 .

In some embodiments, administration of the compounds of the present invention can be to humans testing positive for SARS-CoV-2 but not yet showing symptoms.

In some embodiments, administration of the compounds of the present invention can be to humans upon commencement of symptoms.

In some embodiments, Fe-methisazone and Zn-methisazone, individually or combined together or combined with any other antivirus, can be used for treatment and/or prophylaxis against any virus that has any of the proteins targeted in SARS-CoV2. These targets are spike protein, protease protein and the enzymes responsible for virus replication.

In another embodiment, Fe-methisazone and Zn-methisazone, can be used individually and separately or combined together, or combined with any other antivirus to treat and or prophylaxis against SARS-CoV2 virus.

Fe-methisazone and/or Zn-methisazone can be given intravenously or subcutaneously for the above mentioned indications.

Effective dose of active ingredient depends at least on the nature of the condition being treated, toxicity, whether the compound is used prophylactically or against an active SARS-CoV-2 infection, the method of delivery, and the pharmaceutical formulation, will be determined by the clinician using conventional dose escalation studies. It can be expected that Fe-Methisazone will be administrated orally where adults receive 6 gm a day, taken 3 gm in the morning and 3 gm in the evening while the dose is halved for children between 3 and 10 years old and proportionately reduced still further for infants14. Also, it can be administrated at 200 or 600 mg/kg of body weight15.

Suitable in vitro assays can be used to preliminarily evaluate the efficacy of the fe-Methisazone compound in inhibiting the SARS-CoV-2. The compound can further be examined for its efficacy in treating an infection with SARS-CoV-2. Based on the results, an appropriate dosage range and administration route can also be determined.

One skilled in the art will recognize that substituents and other moieties of the compounds of formula VI should be selected in order to provide a compound which is sufficiently stable to provide a pharmaceutically useful compound which can be formulated into an acceptably stable pharmaceutical composition. Compounds of formula VI which have such stability are contemplated as falling within the scope of the present invention.

### Materials and Methods

### Methods of Identifying Anti-Viral Agents That Interact With SARS-CoV-2 by Virtual Screening

Methods have been developed to identify potential anti SARS-CoV-2 agent using virtual screening. The virtual screening methods identify compounds that may bind to SARS-CoV-2.

### Free Methizasone

The molecular image of the free methizazone was obtained from sketching the structure in the UCSF Chimera molecular visualization software (Pettersen et al., 2004), version 1.14. The hydrogen atoms were added explicitly, and the atomic charges were calculated using Gasteiger charges (Wang, Wang, Kollman, & Case, 2006). Then, the molecular structure was minimized using the Chimera Minimize structure menu command, doing rounds of 5,000 Steepest Descents minimization steps and 100 Conjugate Gradient steps, repeated the needed times until a minimization induces no visible movement in the molecular structure. The resulting molecular coordinates were saved in PDB and MOL2 format (the last one chosen because it also stores the charges).

NOTE: the molecule used as the free Methisazone is a cationic form (formal charge +1) where the terminal sidechain nitrogen is in NH3 form.

### Metal-Bound Methisazone

We decided to build metal coordination complexes with the nitrogens side chain of the Methisazone (branch in the upper right corner of Erreur! Source du renvoi introuvable.a). For placing the metal atom, using the Build structure menu command of UCSF chimera, we manually created a helium atom and placed in the place that we calculated that the complexed metal should be. Then we edited manually the PDB file to change the atom name and element to reflect a zinc atom and to incorporate it into the same model and residue than the uncomplexed Methisazone. As we did with the free Methisazone, for the Zn-Methisazone molecule we calculated the atomic Gasteiger charges and did rounds of minimization until no molecule topology change was detected. For calculating the coordinates of the complexes with iron, cobalt and copper, we took in each case as base the final coordinates of the Zn-Methisazone. All these metal-Methisazone coordinates were saved in the MOL2 format in the Chimera software, and used by the AutodockTools application to generate the PDBQT files that AutoGrid and Autodock. The pdbqt files generated required some manual edits to avoud that the Autodock software crashed with an error message, resulting in the PDBQT files that are in the Supplementary Data.

The dockings were performed with the Autodock 4.2 software(Morris et al., 2009), using the Lamarckian Genetic Algorithm (Morris et al., 1998), with 20 evaluations for run, 25, 000, 000 iterations, and all the other parameters with the default values. For executing the dockings, first are calculated interaction potential grids for the types of atoms that are in the ligands to screen. One of the parameters that one has to choose for that grids is the spacing. At higher spacing, more space comprehended, but less resolution, and viceversa: At higher resolutions, less space comprehended. We at the first used low-resolution potential grids (grid spacing 0.8 Ångstroms) to get a quick image of the binding energies of the Methisazone s (data not shown) but then decided to calculate high-resolution potential grids (0.375 Ångstroms) to get more exact results. For that, we needed to construct many potential grids for each protein, each potential grid overlapping with the adjacent the space needed for the methizasone to float freely (spaced calculated to be 10.811 Ångstroms), and letting that space free (at minimum) around all the faces of the protein surface. For calculating that overlapping potential grids by segments, we calculated the segments positions using a package in Python, whose code created the input files for AutoGrid and for AutoDock, and also submitted the Autogrid and AutoDock runs, and later validated that all the runs finished correctly. Later, we used the script to concatenate all the docking results for the same protein and the same ligand, and to compare results across proteins and among different ligands.

### Results:

First we ran the docking using low-resolution potential grids (with a spacing of 0.8 Ångstroms), but we saw that, for example, the docking results were within 5 Ångstroms of the centers of allostheric sites or structurally important cavities found by the PARS server (Panjkovich & Daura, 2014) (results not shown) so we decided to repeat the analysis using high resolution potential grids with a spacing of 0.375 Å. A simple potential grid does not give enough space for a protein and a surrounding space for the metisazone, so we created a Python (refs) package for calculating multiple partially overlapping potential grids to cover all the protein volume giving space in the overlappings and in the surroundings for the Methisazone to explore freely.

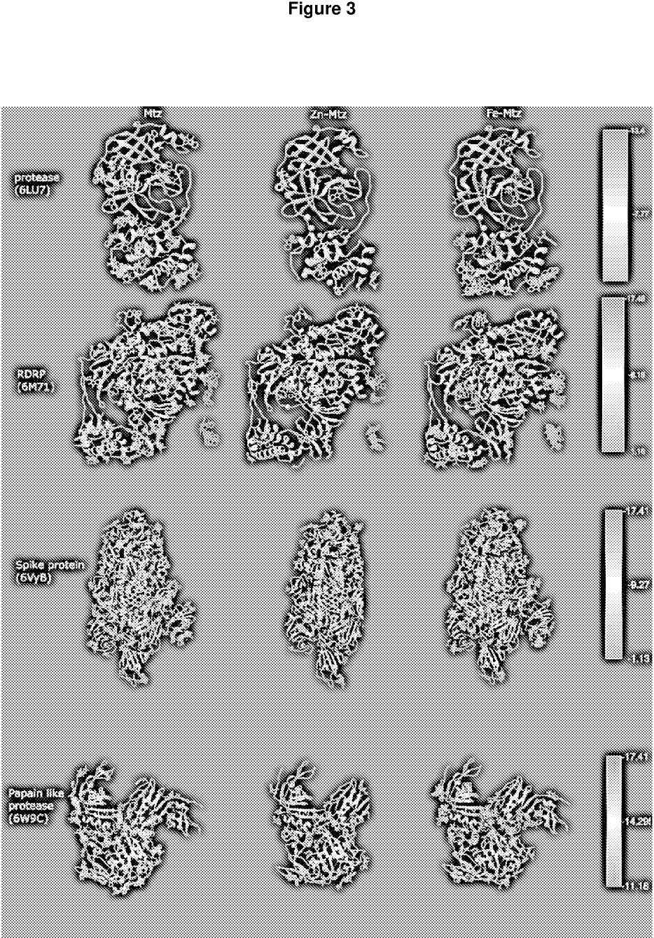

With reference to FIG. 3, Methisazone docked with the SARS-Cov2 proteins main protease, RNA dependent RNA polymerase, spike protein, papain-like protease (PLPro). The Methisazone is in its free state (left), complexed with zinc (center) and in complex with iron (right). The Methisazone molecules predicted positions are displayed as ball and stick, with a color corresponding to their predicted binding energy following the scale of the righ (in kcal/mol). The proteins are shown according to the PDB structures which codes are in parenthsis in the protein label 8at the left), displayed as ribbons.The green balls are the allostheric sites and binding sites predicted by the PARS server

With reference to FIG. 1 Fe-Methisazone docked with the RNA-dependent RNA polymerase (RDRP) of Sars-CoV-2. The Methisazone is in its best position (ranked by energy) which resulted near to a predicted pocket or allostheric site, and which resulted in a similar position that the remdemsivir position resolved by crystallography. The remdemsivir is shown in yellow, and the Fe-Methisazone in blue, following the scale in the bar at the right. Both molecules are shown as balls and sticks. The RDRP is shown .according to the PDB structure 6M71 displayed as ribbons. The green balls are the allostheric sites and binding sites predicted by the PARS server.

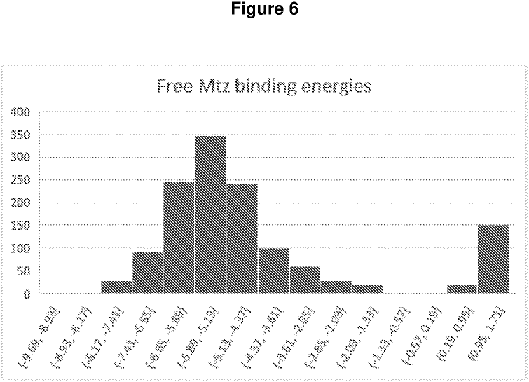

With reference to FIGS. 5-6, comparing with other inhibitors, we have that the remdemsivir has a calculated binding energy of -8.28 ±0.65 kcal/mol with the RNA-dependent RNA-polymerase (RDRP) (Zhang & Zhou, 2020). That binding energy was calculated using molecular dynamics and free energy perturbation. Here we have that the methizazone displays better binding energies to Sars-Cov-2 proteins in its Fe-complexed form, with the better binding result at -17.46 kcal/mol vs the RDRP, at -17.41 kcal/mol of Fe-methizazone vs the spike protein, and -15.96 of Few-methizazone vs PLPro. The better result that lied near the positions predicted by the PARS server to be an allosteheric site or a binding pocket is for the Fe-Methisazone vs the RDRP, with a predicted binding energy of -15.02 kcal/mol.

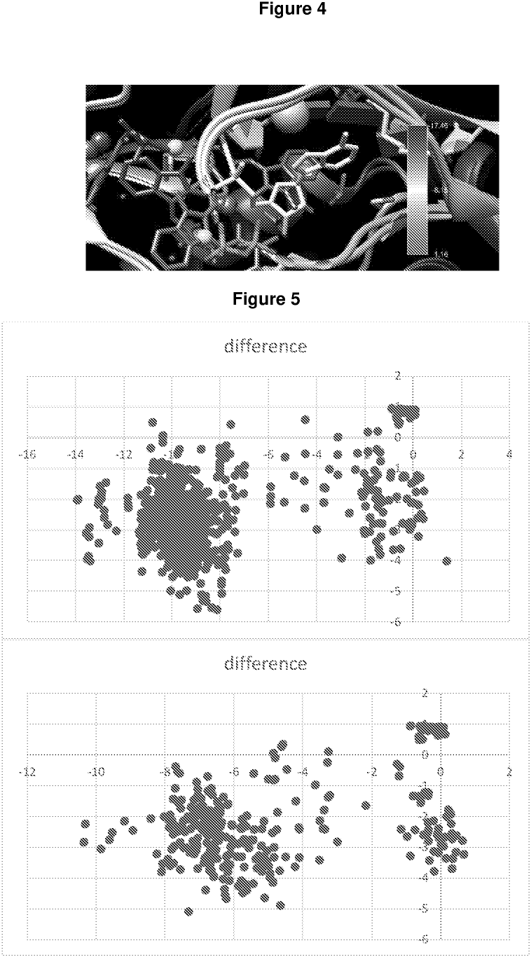

The predicted binding energy of the compounds are largely helped by the bound metals. CONTROL: dock a free iron atom and a free zinc atom. That is to the degree that other contributions looks rather small FIG. 3. That graph shows an average binding energy of -10 kcal7mol for the iron atoms, and a somewhat littler average binding energy of -7 kcal/mol for the zinc. While the other atoms and factors contribute in average with -3 kcal/mol to the binding energy. By other side, the Methisazone without metal shows a binding energy average of -5 kcal/mol (FIG. 4).

The top ranked results resulted of the Fe-Methisazone to the RDRP (-17.46 kal/mol), to the spike protein (-17.41 kcal/mol) and PIPro (-15.96 kcal7mol). Meanwhile, the highest results for zinc started with -13.19, but this time the top 10 results were bound equitatively to all proteins. While the free Methisazone top results started at -9.69 kcal/mol, being bound to the spike protein the top 10 results.

### Acknowledgements

Molecular graphics and analyses performed with UCSF Chimera, developed by the Resource for Biocomputing, Visualization, and Informatics at the University of California, San Francisco, with support from NIH P41-GM103311.UCSF Chimera is developed by the UCSF Resource for Biocomputing, Visualization, and Informatics, supported in part by the National Institutes of Health. Citations are important for demonstrating the value of our work to the NIH and other sources of support.

1. A metal complexed Methisazone or a pharmaceutically acceptable salt thereof, and optionally a pharmaceutically acceptable carrier or excipient for for use in the prevention and/or treatment of SARS-CoV-2 infections.

2. A metal complexed Methisazone or a pharmaceutically acceptable salt thereof, and optionally a pharmaceutically acceptable according to claim 1 wherein the metal complexed Methisazone is an iron complexed Methisazone.

3. A metal complexed Methisazone or a pharmaceutically acceptable salt thereof, and optionally a pharmaceutically acceptable according to claim 1 wherein the metal complexed Methisazone is a zinc complexed methisazone.

4. A method for inhibiting the production of SARS-CoV2 comprising treating a human infected with SARS-CoV2 with a pharmaceutical composition comprising a therapeutically effective amount of metal complexed-Methisazone or a pharmaceutically acceptable salt thereof, and optionally a pharmaceutically acceptable carrier or excipient.

5. A method according to claim 4 wherein the metal complexed-Methisazone is an iron complexed Methisazone .

6. A method according to claim 4 wherein the metal complexed Methisazone is a zinc complexed Methisazone .
